(19) United States
(12) Patent Application Publication
(10) Pub. No.: US 2006/0110297 A1
(43) Pub. Date: May 25, 2006

(54) Vehicle cabin air filter freshener

(76) Inventors: Daniel D'Amico, South Salm, NY (US); Daniel Grewe, Grove, IL (US); Neal Harris, Los Angeles, CA (US); Patrick Gerring, Lexington, KY (US); Jeremy R. Brookes, Lexington, KY (US); Brenda L. Arnold, Lexington, KY (US)

Correspondence Address: David W Carrithers;Carrithers Law Office, One Paragon Centre, Suite 140 6060 Dutchman's Lane, Louiseville, KY, 40205, US

(21) Appl. No.: 10/544,157

(22) PCT Filed: Aug. 13, 2003
(86) PCT No.: PCT/US03/25494

§ 371 Date: Jan. 17, 2006

Related U.S. Application Data
(60) Provisional application No. 60/466,224, filed on Apr. 28, 2003.

(51) Int. Cl. A61L 9/12

(52) U.S. Cl. 422/124

(57) ABSTRACT

An air freshener filter insert for use with the air filter in a motor vehicle air conditioning, air ventilating systems. The air freshener filter insert is a strip of permeable material such as a mesh which may be formed having pockets spaced apart from one another longitudinally there along. The pockets contain a selected quantity of a solid particulate material impregnated with a time release substance of selected fragrance. A length portion of the air freshener strip is cut to match the length of a replacement air filter and then mounted on a face thereof. The combined unit is then installed in the vehicle ventilating system. Servicing of the air filter can readily be done at quick lub service stations.

This application claims priority from U.S. Provisional application Ser. No. 60/466,224 filed on Apr. 28, 2003 which is hereby incorporated by reference in its entirety.

### FIELD OF INVENTION

The present invention provides a high efficiency disposable air freshener filter strip insert having an air freshening substance releasably retained in an air flow through particulate or porous material held captive by means for holding such as a porous mesh, (disposed within or nested together) with a conventional cabin particulate air filter. The unit is readily positionable in the airflow passage between the respective inlet and outlet(s) therefor of a vehicle cabin air circulating system, preferably on the downstream side of the air flow through filter. The disposable air freshener strip insert is designed to fit in the existing filter housing of an air flow ventilation system together with a conventional particulate air filter.

### DESCRIPTION OF THE PRIOR ART

Motor vehicles commonly are provided with a heating-cooling air ventilating system (HVAC) for the occupants in the cabin of the vehicle. Conventionally there is an air inlet exterior of the vehicle occupant compartment(the cabin) and outlets that are strategically located for distribution of the air within that compartment. At least some of the components of the system are mounted on the firewall between the engine and occupant compartments and air is directed to flow through a coil that heats or cools (as the case maybe) air that flows there through.

In recent years one or more replaceable air filters have been added to the air circulating systems to remove air borne particles and thus improve the quality of the air for the occupant(s) of the vehicle. Particulate filters are designed to trap small particles such as dust and in some cases pollen. In some instances carbon filters are used for removing odors from the air in or entering the cabin. The carbon filters may also be used in combination with the particulate filters and may even be a dual layer filter. In many cases filtering of the air is necessary because of allergies and/or medical conditions of occupants in the vehicle. The filters are variously located depending upon the make and model of the vehicle. In some instances they are located above the accelerator and in others behind the glove compartment box while others are under the hood in the motor compartment. Irrespective of location they are obviously placed in the air flow passage such that the air must flow there through.

In most automobiles, the air filter within the outside air intake is located under the hood, behind the glove box, or under the dash and either above the blower in flow communication with the outside air intake or between the blower and the HVAC assembly.

The air intake filters can be replaced by opening the hood and removing the air intake screen held in with plastic fasteners. If a rain deflector is disposed over the filter remove it. The filter element should be exposed and can be removed from the filter housing which can be removed with any gasket which may be used to provide a seal thereinbetween.

The air intake filters which are disposed in the housing in close proximity and flow communication with the blower are usually located above the blower behind the glove box. The glove box may have to be removed to access the filter housing.

In some cars, a midcase filter may contain a plurality of side-by-side filters disposed within a cartridge whereby the cartridge may be removed from the HVAC housing providing access to the filters and gasket or other means for sealing the filters within the cartridge.

In addition to the vehicle built in air circulating and/air conditioning (HVAC) systems there are after market air circulating systems that can be placed within the occupant compartment of the vehicle and which have a filter for filtering out the air borne pollutants and/or removing odors (such as) using a charcoal filter disposed adjacent (with) the particulate filter or a filter composed of conventional foam, plastic or paper having active charcoal particles embedded therein to remove odor by adsorption. Some filtering systems meet HEPA standards.

The conventional cabin air filters known in the art may be formed from a flat piece of porous material, a flat layer of foamed material such as rubber, or a pleated material such as an air porous paper product which includes a paper or plastic frame there around or a gasket for sealing the edges of the filter against the filter housing or adjacent filters. Moreover, the filter may be electrostatically charged. Pleating of the filters increases the surface area.

It is believed an occupants well being and/or state of mind can be enhanced by having an air filter freshener device that provides a pleasant smell. There are many known different fragrances available and one can be chosen that meets the needs and/or requirements of an individual or vehicle manufacture or an establishment that services vehicles as the case maybe. Vehicle servicing, manufacturing and/or vehicle selling establishments may choose a fragrance that is peculiar or distinct to their establishment and over time may become their trademark in business.

U.S. Pat. No. 6,309,715 sets forth several patents which describe methods of incorporating a slow sustained release of a fragrant molecule as follows: U.S. Pat. Nos. 5,525,588; 5,525,555; 5,490,982; 5,372,806 describe dissolving or suspending fragrance compounds in emulsions. U.S. Pat. Nos. 5,500,223; 5,324,444; 5,185,155; 5,176,903; and 5,130,171 describe encapsulation of a flagrance. U.S. Pat. No. 5,234,689 describes dissolving a fragrance into a hydrophilic phase such as silicone; U.S. Pat. Nos. 5,387,622 and 5,387,411 incorporation of a fragrance into a cross-liked polymer. U.S. Pat. Nos. 5,071,704 and 5,008,115 incorporate a fragrance into a permanent laminate. U.S. Pat. No. 4,908,208 incorporate a fragrance that softens at body temperature. U.S. Pat. Nos. 4,524,018 and 4,500,725 describe incorporation of a fragrance into silanes with fragrant alcohol to form alkoxysilanes. U.S. Pat. No. 6,054,547 describes the incorporation of fragrant moieties via hydrosilation of an olefinic silane molecule. U.S. Pat. No. 5,034,222 describes a solid composite air freshening article in a granular foam phase dispersed throughout a gelled phase. U.S. Pat. No. 6,063,365 describes an emulsifier-free nonporous, continuous permeable polymeric film having an entrapped and dissolved fragrance capable of evolving from the film into the environment by means of molecular diffusion in a sustained and controlled release manner. The disclosure of the above U.S. patents are hereby incorporated by reference herein as if set forth in their entirety.

An air freshener strip filter insert generally consists of a substrate impregnated with a substance that is time released into the surrounding air and in which such substance, when in the air, can, upon appropriate choice, provide a pleasant aroma. Air fresheners are available that are attachable to the grill of an air outlet of an air ventilating system so as have the air freshening substance added directly to the air flowing into a vehicle cabin or they maybe passive and merely consist of a substrate or carrier containing an aroma producing substance and fixed or attached to some part of the vehicle within the occupant compartment.

Maintenance of vehicles includes changing the oil, oil filter and/or the motor air filter from time to time and the frequency thereof depends upon such factors as type of use, time of use and conditions in which the vehicle is used. A cabin air filter should also be serviced from time to time.

While vehicle maintenance is normally on a regular basis vehicle air filter fresheners seem on the other hand to be given a low priority with replacement being done at the will of the operator and most often ignored completely. A pleasant aroma however can have a soothing affect on the occupants and in this day and age of frequent happenings of road rage the soothing of nerves can not only be for the well being and comfort of the occupants but also beneficial from a safety point of view. Accordingly, there should not only be an air freshening element but one that is serviced periodically and systematically for example when the vehicle is taken in for other servicing such as an oil change.

### SUMMARY OF INVENTION

The present invention provides a method and means for incorporating a high efficiency disposable unit having an air freshening substance releasably retained in an air flow through particulate or porous material held captive by a porous mesh, (disposed within or nested together) with a conventional cabin air filter. The unit is readily positionable in the airflow passage between the respective inlet and outlet(s) therefor of a vehicle cabin air circulating system, preferably on the downstream side of the air flow through filter. Some air filter systems have a carbon filter to remove odors from the air in which case the air freshening device must be located on the downstream side of the carbon filter.

A principal object of the present invention is to provide a replaceable air freshening device for an occupant compartment air circulating system of a motor vehicle.

A further object of the present invention is to provide an air freshening device that is readily customized to fit various different air circulating systems and particularly ones having an air flow through filter therein

Another object of the present invention is to provide an air freshener device having high surface area capable of being disposed within the pleats of a conventional particulate filter to maximize the surface area of the fragrance filter and utilize the structural support of the conventional pleated particulate filter.

It is a further object of the present invention to provide a deposible flagrance cabin air filter means wherein a particular scent or aroma can be distributed throughout the (ventilation and) ventilated cabin area and provide a means for aroma therapy with selected scents including fruit and berry scents, such as evergreen scents, citrus scents, vanilla, and spice scents.

It is a further object of the present invention to provide a disposable flagrance cabin air filter means wherein a disinfectant, deodorant, biocide, deodorant, bactericide, fungicide, mold, and/or mildew retardant or the like can be distributed throughout the ventilation and cabin area in order to purge the ventilation system and be removed or retained in an amount to retard contamination by same.

A further object of the present invention is to provide a disposable air freshening medium that is long lasting and subject to replacement during an oil and oil filter change.

It is another object of the present invention to utilize an assembly utilizing the control system and air conditioning-heating-circulation (HVAC) system including the fan, plenum, conduits, vents, and existing air filter housing assemblies and filters wherein an air filter freshener element disposed within the filter housing comprises of one or more of solid particles, strips, or bars, impregnated with a substance that is time released into the surrounding air and in which such substance when in the air can upon appropriate choice provide a pleasant aroma without requiring removal of the conventional air filter.

These an other objects and features of the invention will become apparent to those skilled in the art from the following detailed description and appended claims.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 is an oblique view of an air filter for an automobile heating, ventilating, air conditioning system;

FIG. 2 is a side edge view of a strip portion of an air freshening element provided in accordance with the present invention for use in association with a filter in a vehicle cabin air ventilating system as for example the filter illustrated in FIG. 1;

FIG. 3 is a bottom face view of the air filter freshener element shown in FIG. 2;

FIG. 4 is an edge view showing an alternate embodiment of a fragrant filter;

FIG. 5 is a bottom face view of the air freshener element illustrated in FIG. 4;

FIG. 6 is an edge view of an air freshener element constructed in accordance with the embodiments shown in FIGS. 2, 3, 4, and 5 and including an outer protective wrap formed of a mesh or other air permeable material such as a polymer overwrap;

FIG. 7 is an edge view of a vehicle air vent system filter and a rolled up supply of an air freshener filter insert strip of the type illustrated in FIGS. 2, 3, 4, and 5 which diagrammatically illustrates the procedure for custom fitting a selected length portion of the air freshener to the filter at hand;

FIG. 8 is an enlargement of a portion of the air freshener filter strip insert of FIG. 2 illustrating the pockets as having been separately formed and then attached to the base strip;

FIG. 9 is an enlargement of a portion of the air freshener filter strip of FIG. 2 showing the base strip being folded and joined as by welding or other suitable adhesive to form envelopes at spaced apart intervals;

FIG. 10 is a side view of an alternate embodiment of a cabin air system freshener wherein the fragrance is contained with a polymer cut into strips or bars attached to an air permeable mesh strip or other flow through material;

FIG. 11 is an alternate embodiment showing an air freshener filter insert strip in the form of a film or thin strip of material intermediate a mesh or other air permeable support material such as a plastic grid, perforated paper, plastic, or fabric;

FIG. 12 is a partial, enlarged cross-sectional, view of the filter shown in FIG. 1 together with an air freshener filter insert strip including a base strip and a plurality of tabs extending therefrom and projecting between selected pleats of the fibrous particulate air filter;

FIG. 13 is an oblique view of a portion of an automobile with the hood raised and a portion of the vehicle air vent system intake and plenum with the cowl cover peeled back showing the deflector wherein a conventional particulate filter cartridge is pulled out of a drawer and showing a fragrance filter disposed on the underside (downstream air flow side) of the pleated particulate filter whereby the protrusions from the fragrant filter project into the pleats;

FIG. 14 is an oblique view of a HVAC filter housing and blower assembly for an automobile heating, ventilating, air conditioning system showing a conventional particulate filter consisting of a fibrous pleated filter supported within a cartridge wherein the fragrant filter of the present invention is disposed adjacent to and downstream therefrom with respect to the air flow;

FIG. 15 is a perspective view of an under the dash air filter assembly showing an embodiment of the present invention disposed within the pleats of the particulate filter member and showing the fragrant filter having a portion folded upward to show the fragrant element disposed within the pleats of the particulate filter on the downstream side of the air flow;

FIG. 16 is another embodiment of an under the hood filter wherein the particulate filter is positioned vertically within the cowling and an embodiment of the present invention is nested therewith having a portion folded to show the fragrant element on the downstream side of the air flow; and

FIG. 17 is an oblique view of an air filter cartridge containing fiber and foam particulate filters for an automobile heating, ventilating, air conditioning system wherein the porous foam filter is shown having solid particles of an air freshener filter insert impregnated therein whereby the fragrant absorbing material has absorbed a time release substance of selected fragrance.

### DESCRIPTION OF PREFERRED EMBODIMENT

The term air freshener as used throughout herein is intended to be generic to fragrance producing substances as well as disinfectants and the like that can be transferred to a flowing stream of air so to improve the quality of such air.

The air freshener and the present invention is a full air flow through device consisting generally of a substrate impregnated with a substance that is time released into the surrounding air and in which such substance when in the air can upon appropriate choice provide a pleasant aroma.

A particular scent or aroma can be distributed throughout the ventilation and cabin area and provide a means for aroma therapy with selected scents and fragrances. The fragrance oil can be any natural substance, synthetic material, (incorporating aldehydes, ketones, esters, and other chemical constituents), or combinations thereof which is known in the art and suitable for use in candles for imparting an odor, aroma, or fragrance. Suitable natural and synthetic fragrance/flavor substances include those compiled by the U.S. Food and Drug Administration in Title 21 of the Code of Federal Regulations, Sections 172.510 and 172.515 respectively. Suitable fragrances include spice oil, flower oil, and fruit oil. The fragrance oil may contain fragrance components, for example benzaldehydes, phenols, cinnamic aldehydes and esters, octadienes, dienes, cyclohexadienes, and terpenes.

The fragrances may comprise an essential oil in a carrier such as water and/or alcohol or other organic solvent or even a perfume. The fragrance may be that of a fruit and berry scents such as: citrus, almond, apple, cherry, grape, pear, pineapple, orange, strawberry, raspberry, and musk; flower scents such as lavender, rose, iris, carnation, gardenia, tea rose, violet, hyacinth, magnolia, mimosa, honeysuckle, jasmine, narcissus, orange blossom, orchids, sweet pea, tuberose, and lilac; forest and herbal smells such as evergreen cedar, pine, sassafras, and spruce; essential oils such as spice, peppermint, vanilla, spearmint; and various other fragrances such as leather, new car odor, acacia, cassie, cypre, cyclamen, fern, hawthorn and the like. The fragrance is not critical so long as it is compatible with the polymer impregnated with the time release carrier. The level of fragrance can vary up to about 100 percent by weight and more preferably from 0.001 to about 100 percent by weight. Other agents such as surfactants, emulsifiers, and polymers can be used to encapsulate the fragrance.

The carrier and means of controlling the release of the selected fragrance is dependent upon many variables including time and temperature with respect to the environment inside and outside of the vehicle. The fragrance and substrate composition comprising of 10% to 99.5% by weight of a suitable substrate and from 0.5% to 90% by weight of a fragrance oil.

It is also contemplated that the scented material may be composed of 100 percent of the fragrant raw material without a solvent or essential oil. Furthermore, the fragrance can be imparted to the air freshener filter absorbent material as an aerosol or gas vapor separate from, or in combination with a liquid substrate.

In one preferred embodiment, the fragrance can be absorbed by a polymeric material such as an ethyl vinyl acetate polymer simply by being disposed in close proximity thereto. Ethyl vinyl alcohol resin is another polymer having good scent absorbent capabilities.

One preferred group of copolymers of ethylene and vinyl acetate which absorb odors are produced by DuPont Industrial Polymers under the trade name of ELVAX resins which generally range in vinyl acetate content from 9 to 40% and have melt indexes from 0.3 to 500 dg/min. Moreover, the ELVAX resin from DuPont does not incorporate a plasticizer which could interfere with the scent absorbing capability of the polymer. The polymer can be formed in any desired shape prior to treatment or under low heat conditions (70° C. or less). The ELVAX resin can be extruded or molded and blended with polyethylene, polypropylene, ABS resins, thermoplastic rubber nitril rubbers, natural rubber and other elastomers.

The amount of scent adsorbed into the vinyl acetate substrate and the control release mechanism is dependent upon the time and temperature relationship between the scent and substrate as well as the volatility of the scent, content or density of the polymer, level of polymerization, surface area (porosity), and pore size and structure of the polymer substrate which in the instant example is a vinyl acetate absorbent material. Thus, the impregnation occurs through passive adsorption and the time release mechanism occurs through passive diffusion.

As described in U.S. Pat. No. 2,169,055 by Overshiner et al., a cellulose compound such as cellulose acetate or cellulose nitrate can be imparted with a compatible scent imparting material which is released over an extended period of time. The cellulose compound can be produced in a solution with an organic solvent such as acetone and 1,4 diethylene oxide, and adding a scent imparting essential oil to the solution. Plasticizers such as diethyl phthalate and tri-acetic acid ester of glycerin may be used to impart flexibility to the material. The solution may be formed in to sheets, bars, films or the like by casting or molding. The solvents evaporate and a porous cellular structure is formed which releases the essential oil at a rate to provide an odor lasting for several months. Moreover, as shown in FIG. 6, the scent impregnated polymer is over wrapped and crimped at desired intervals to form an air tight seal utilizing a nonpermeable vacuum sealed material to prevent premature release of the scent in storage. Thus, the product can be air evacuated, or even vacuum sealed, in order to prevent release of the scent from the polymeric substrate prior to installation. The strength of the scent may also be determined by the quantity of the individual polymer units, (bars or particle containing pockets), which are cut from the roll and disposed in the filter system. Thus, the customer can decide upon the strength of the air freshener filter insert by using multiple packs cut to length. The utilization of sealed polymer substrates containing scents provides a method of prolonging and maintaining the shelf life and consistency of the time release product.

Micro encapsulation provides a method of controlling the release of scent in liquid form by enclosing the scent within hollow shells of differing size and wall thickness which can be dissolved or ruptured at different intervals to provide a generally steady supply of scent exposed to the environment. Moreover, semipermeable shells which allow escape through the shell wall without shell rupture exist to control the release of a scent.

Another method of controlling the release of the scent from a carrier is to use a homogeneous semipermeable material containing the active ingredient as a pure impregnate, solute or precipitate. The semipermeable material serves as the carrier from which it can only slowly escape by solution, diffusion, evaporation or combinations thereof. The characteristics of the carrier material depends on properties such as pore size, compatibility with the environment, liquid content, temperature of environment, wet-ability, and processing parameters.

One type of polymer liquid composite material prepared which can be utilized in the present invention is an organic or inorganic cellulose ester such as cellulose triacetate or cellulose nitrate as vehicles for the controlled release of active materials into the environment. As described in U.S. Pat. No. 3,985,298 by Nichols and incorporated by reference herein, the composite material can be formed to prepare transparent coherent materials formed as films, fibers or microspheres. The scent may be carried in a fluid which may comprise water, alcohol, ether, aliphatic and aromatic hydrocarbons, ketones, esters, and combinations thereof together with other chemical constituents. For instance, the chemical composition comprising the selected fragrance or scent or other chemical such as a disinfectant or mildew or fungicide may be incorporated as a component in an aerosol propellant, gas, or liquid containing a solvent carrier such as water and/or alcohol together with an essential oil having a selected fragrance or perfume for impregnation into the polymer-liquid composite. These polymer-liquid composite materials are prepared to incorporate interconnected internal pores from about 1 to 500 microns. These polymer-liquid composites often possess oleophilic hydrophobic surfaces and can contain release retarding gums and oils from which hydrophilic and oleophobic vehicles can be expelled by surface forces. Moreover, active ingredients can be incorporated in polymer-liquid composites as soluble particles or precipitates formed by solvent exchange or chemical reaction in situ to provide an internal reservoir which maintains a constant concentration of the active ingredient in solution inside the polymer-liquid composite. Thus, the polymer-liquid composite provides a means for zero-order release in which a nearly uniform level of active ingredient (scent) is maintained throughout the active life of the vehicle. The polymer-liquid composite material provides a means of retarding the evaporative release of volatile materials though diffusive effects and control of effective surface area, as well as through depression in the vapor pressure of the volatile substance. Furthermore, the polymer-liquid composite can be modified by skinning, by coating with a liquid to impede escape of the active ingredient (scent) or by the addition of a release-promoting agent to the environment.

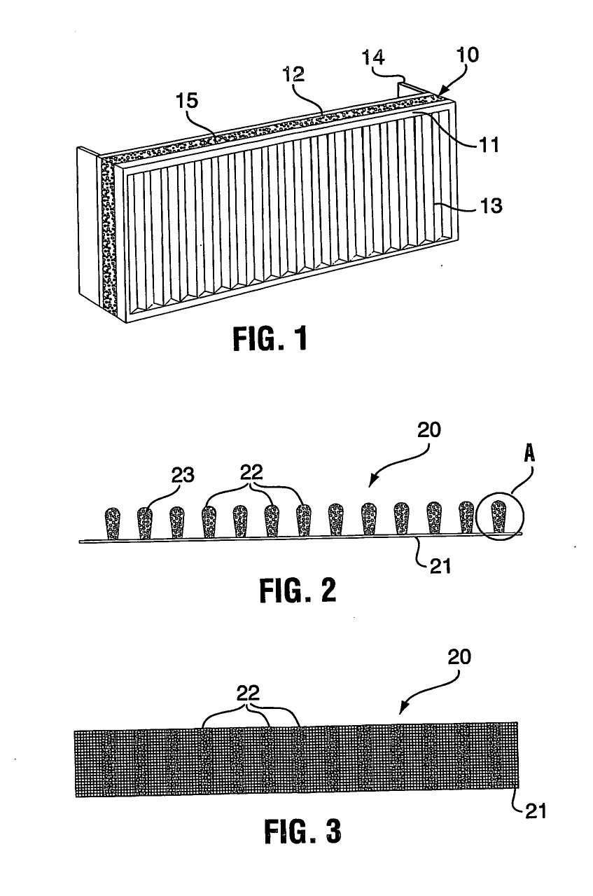

Illustrated in FIG. 1 is an air filter and more particularly an air filter 10 that has been removed from the air passage of a motor vehicle air conditioning and ventilating system which conventionally heats or cools, as the case maybe, the cabin of a motor vehicle and/or circulates the air. The filter removes air borne particles from the air and in some cases includes activated carbon to remove odors. The filter illustrated has a center layer 15 of porous foam sandwiched between respective outer layers 11 and 12 each of which is made of fibrous filter material which may be composed of paper, cloth, synthetic material and combinations thereof. Although not required, conventional filters typically utilize a fibrous material pleated as indicated by the lines designated 13 to maximize the surface are of the filter element. These conventional air filters may be constructed of various materials and methods; however, the construction thereof is not of concern with respect to the present invention other than if containing a substance to absorb odors. The filter 10 illustrated has flanges 14 secured thereto and projecting outwardly from one face adjacent opposite ends of the filter for use in positioning the filter in the air circulating system. The flanges 14, although not essential, also provide something to grip when removing or replacing the filter 10.

In accordance with the present invention, there is provided a vehicle cabin air flow through strip having an air freshener filter insert strip or element 23 contained therein. The strip 20 is for use in association with a vehicle's conventional air filter of the vehicle's air conditioning and ventilating system. As best illustrated in FIGS. 2 and 3, the air flow through strip 20 comprises a base strip 21 of porous material having a plurality of pockets 22 attached thereto. The pockets 22 are spaced apart from one another longitudinally along the strip 21 and each holds captive therein a selected quantity of scented particulate material 23. The base strip 21 and pockets 22 are preferably made of a mesh material allowing for air to flow readily there through. For example, the mesh may be fabricated from paper, cloth, synthetic material and combinations thereof such as fiberglass, nylon, polyester, polyethylene or the like. Moreover, a fibrous material or film including openings therein may also be substituted for the mesh. The scented particulate material comprising the fragrant element is composed of beads of a polymer such as an acetate material impregnated with a substance having a preselected scent. The porosity of the material is obviously co-related to the particle size of the particulate material so as to retain the particulate material on the base strip 21.

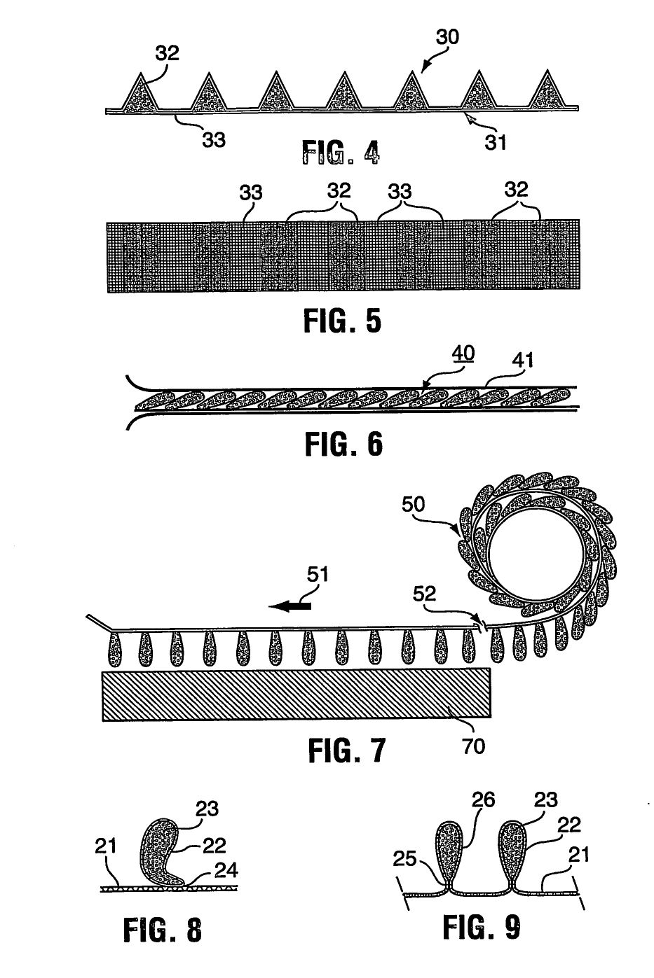

In the foregoing embodiment, the pockets 22 are separately formed and suitably attached to the base strip as for example by stitching and/or a suitable adhesive or fusion bonding using heat and/or suitable solvents. In the embodiment illustrated in FIGS. 4 and 5 the base strip is suitably folded to provide pockets that can be filled with a selected quantity of fragrance impregnated (beads) acetate beads or the equivalent. Moreover, flakes, strips of material, or solid bars may be used to form bars, strips, or wedges which may be disposed within the pockets 22. The base strip 21 is a porous mesh and maybe a single strip suitably folded to provide pockets 22 or there maybe two strips 21 overlying one another and at least one strip being suitably folded and the strips joined together to provide pockets 32 that are spaced apart from one another longitudinally along the length of the strip 31. The base strip material, as set forth in the previous embodiment, is a porous mesh.

Referring to FIGS. 4 and 5 there is illustrated an air freshener filter insert 30 comprising a base strip 31 suitably folded and joined together to provide alternate pocket filled areas 32 and open mesh areas 33. The pockets 32 contain a quantity of particulate material or equivalent impregnated with a substance that provides a selected fragrance.

FIG. 8 is an enlargement of the encircled portion “A” of FIG. 2 illustrating the pockets or envelopes 22, with such envelopes being separately formed members and attached as by fusion bonding or the like as indicated at 24 to the base strip 21 made of an open mesh material.

FIG. 9 is the same as FIG. 8 but showing the base strip fusion joined as indicated at 25 at space apart intervals and at each fusion bond the strip is folded upon itself to provide a loop 26 in the strip to form the pockets 22. The edges of the loops are sealed thereby providing an enclosed envelope for confining a selected quantity of fragrance impregnated beads or the equivalent thereof.

Illustrated in FIG. 6 is an air freshener filter strip insert 40 utilizing the air filter freshener element 20 shown in FIGS. 2 and 3, that maybe of the construction illustrated in FIGS. 2 and 3 or in FIGS. 4 and 5, or the equivalent thereof, and having an over wrap 41 which facilitates spooling, shipping, and storage of a roll of the air freshener filter insert strip 40. The over-wrap maybe a polymer material which may be removed before cutting a suitable length of the air freshener filter insert strip to be used with the filter at hand, or it maybe a porous material and remain on the portion of the strip removed for use in the task at hand.

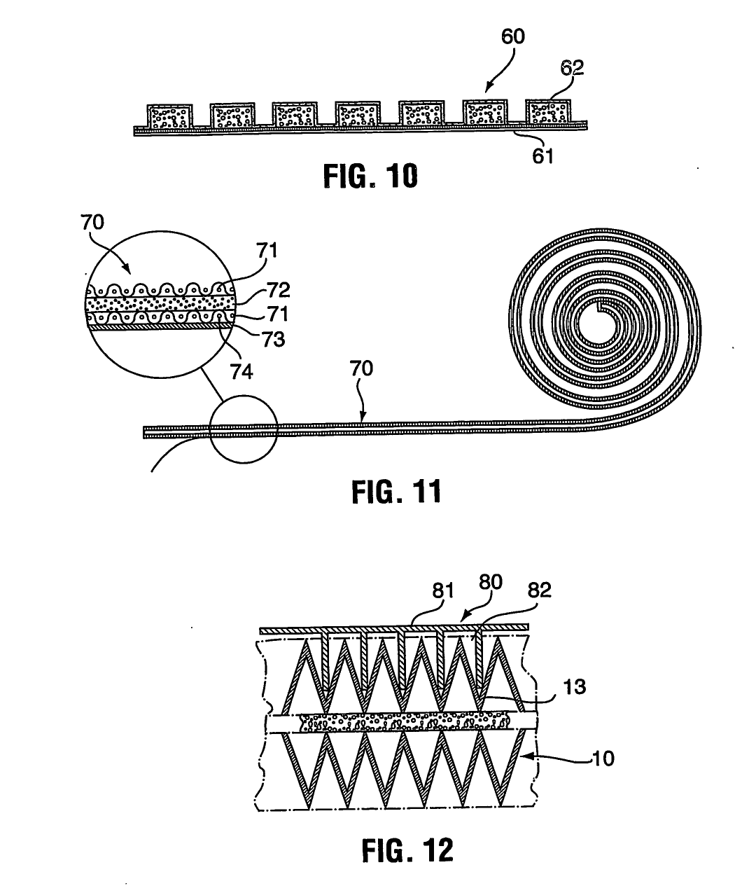

Shown in FIG. 10 is an alternate embodiment of an air freshener filter insert strip 60 that includes one or more scented elements 62 mounted thereon is spaced apart relation. The scented elements 60 maybe strips, blocks, bars, tabs or the like adhering to and projecting a short distance from the filter strip 61. The fragrant elements 62 are molded or otherwise formed of a scented material and typically comprises a porous material such as an acetate polymer impregnated with a scent. The freshener element 62 may be enclosed in a porous means for holding such as a pocket or the like. It is also contemplated that the fragrant element may be formed of a soft pliable material such a highly plasticized polymer or material held in pockets or other means of retaining forming a frame in order to provide a means for inter nesting the fragrant elements in the pleats of a conventional pleated air filter. The fragrant material can be contained in bags as shown in FIGS. 1-9 or affixed to a support backing of permeable material such as a web, frame or other permeable material such as a sheet of plastic having openings there through by molding, glueing, or impregnation by melting thereto. As shown in FIG. 10 the scented element 62 is affixed to a synthetic mesh backing. An important consideration is to provide as much surface area as possible to maximize the exposure of the air to the air freshener filter insert element.

Referring to FIG. 7 there is illustrated a supply roll 50 of an air freshener filter insert strip constructed as illustrated in FIGS. 2 and 3 or in FIGS. 4 and 5, or the equivalent thereof. A length portion of the strip is payed out from the roll in the direction of arrows, measured as diagrammatically to match the length of the vehicle air filter 70 and cut as diagrammatically indicated at 52. The cut length portion of air freshener filter insert strip is then placed to overlie a face of the filter 70 and attached thereto as for example by portions of a double faced tape or an adhesive both of which are preferably removable or merely interleaved with the pleated outer face of the air filter. The combined unit is then inserted into the vehicles air vent system. In the event the filter 70 is or has a carbon portion for removing odors from the air the air freshener filter insert strip must then be placed on the airflow downstream side of the filter 70.

Described in the foregoing is an air freshener filter insert strip for an air filter in which the fresher strip is formed from a mesh type material that is for example a polyester, polythene, nylon, fiberglass or the like. Beads of for example of an acetate that have been impregnated with a time release fragrance are retained in selected areas spaced apart from one another longitudinally along the length of the strip. The air freshener filter insert strip can be rolled up with lengths cut therefrom as needed to fit the air filter or frame that holds the same. With the freshener strip overlying a face of the filter-in-situ in a motor vehicle heating, ventilating, cooling air ventilating system air can flow readily though the freshener strip in a plurality of flow paths and flow past the face and to some extent through the pocket, i.e., envelope containing the fragrance impregnated beads or other suitable fragrance retaining material.

FIG. 11 is an alternate embodiment of the present invention showing an air filter freshener 70 including an air filter freshener element 700 comprising a porous film or thin strip of material having openings therein sandwiched between two layers of a base strip 71 comprising a mesh or other air permeable support material such as a plastic grid, perforated paper, plastic, or fabric disposed on each side of the air filter freshener element. Optionally, a thin layer of a weak adhesive can 73 can be disposed on a selected surface of the support material and covered with a removable and/or peelable film or paper backing 74. The adhesive can serve to affix the fragrance strip to the air filter. The adhesive may also be applied to one side of the removable film or paper 74 and applied to the roll of air freshener filter insert strip 70 during the fabrication of same.

Illustrated in FIG. 12 is an air freshener filter insert strip 80 on one face of a conventional air filter 10 shown in FIG. 1. The air filter is shown as a partial, enlarged, cross-sectional view. The air freshener filter insert strip 80 comprises as base strip 81 from which projects a plurality of short tabs or bars 82 that are spaced apart from one another longitudinally along the base strip 81. The width of the tabs 82 corresponds preferably to the width of the original air filter 10 in the housing of the HVAC system of the vehicle and the tabs project into the valley of two adjacent pleats 13 of the pleated conventional filter 10 for the air conditioning system. The tabs 82 may comprise a bag as shown in FIG. 2 containing a quantity of particulate material impregnated with a time release substance of selected fragrance or they may simply be a felted or other type of material that, over time, releasably retains the fragrance producing substance. The base strip 81 may also be of the same width as the filter and made of porous material or alternatively made of a plurality of narrow strips or strings spaced apart from one another across the width of the filter. The spacing between the narrow strips would allow free flow of air. The tabs projecting into the filter, i.e., between the adjacent pleats keeps the air freshener device mounted to or inter-nested with the air conditioning system air filter.

The air freshener filter insert strip of the present invention can be installed in vehicles as easily as if replacing the original air filter.

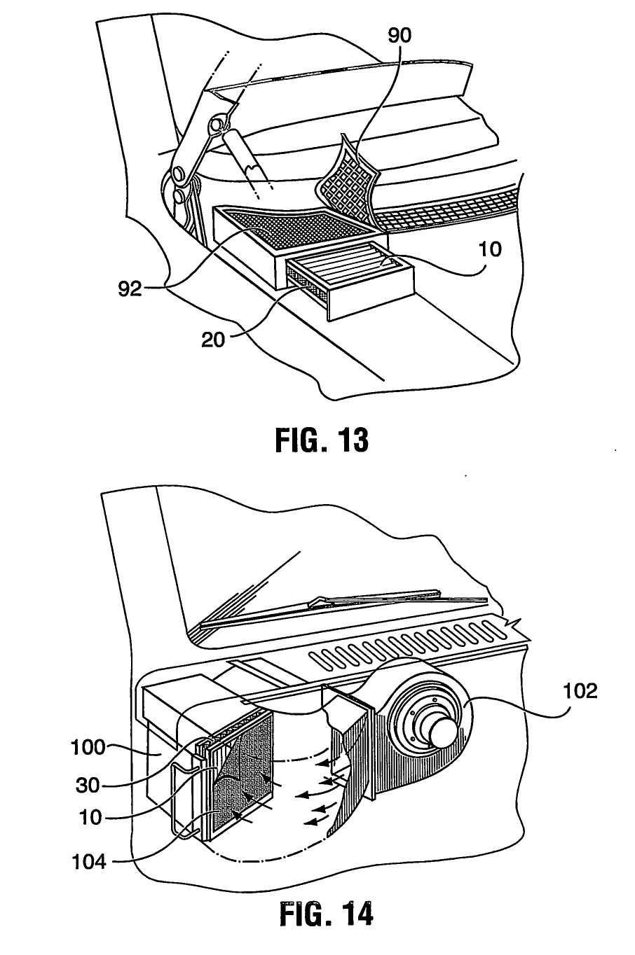

For instance, FIG. 13 illustrates a portion of an automobile having a raised hood showing an air intake and plenum with the cowl cover 90 peeled back. The air filter cartridge 10 is in a drawer shown pulled out and a fragrance strip 20 is shown disposed on the underside (downstream air flow side) of the pleated air filter. The air freshener filter insert strip maybe as disclosed in the foregoing with protrusions carrying the fragrance portion projecting into the pleats.

FIG. 14 shows an alternate filter arrangement comprising a filter housing 100 and fan blower 102 assembly of an automobile heating, ventilating, air conditioning system. A conventional pleated air filter 10 is supported within a cartridge in the plenum. A fragrant freshener strip 30 of the present invention is disposed adjacent a face of the filter on an air flow downstream of side thereof. A carbon filter 104 is disposed upstream of the air filter and adjacent thereto.

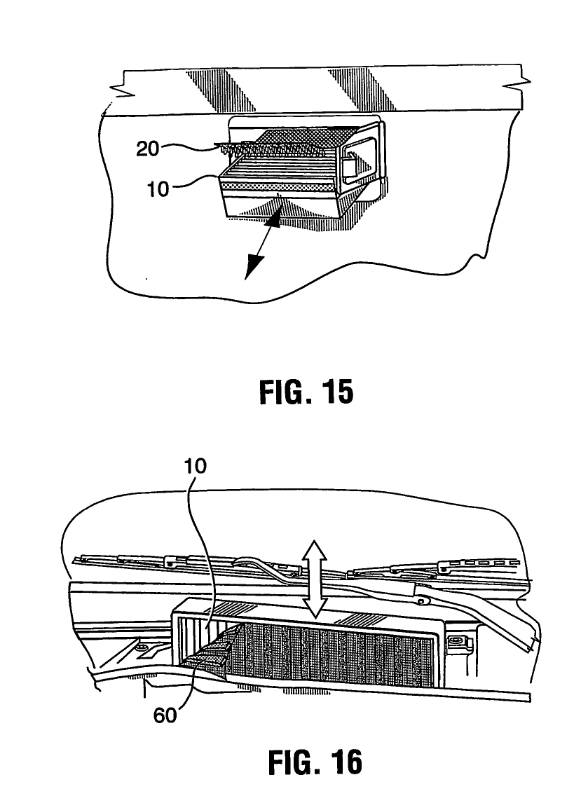

FIG. 15 illustrates another method of mounting a filter and housing assembly under the dash of a vehicle. The dash air filter assembly shows an embodiment of the present invention wherein an air freshener filter insert strip 20 includes a plurality of pockets filled with beads impregnated with a time release fragrance. The pockets nest in-between at least some of the pleats 13 of the air filter 10. The air freshener filter insert strip 20 is illustrated having a portion folded upward to show the pleats of the air filter.

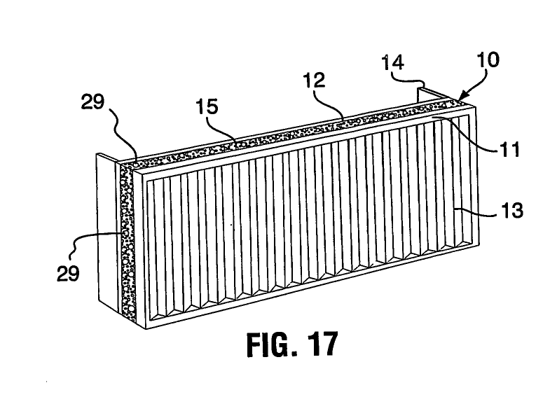

FIG. 16 is another embodiment of an air freshener filter insert strip 60 showing an under the hood filter housing incorporating an air filter 10 positioned vertically within the cowling and an embodiment of the present invention having bars or tabs 62 as fragrant elements nested with pleated air filter surface. A portion of the air freshener filter insert strip is folded to show the fragrant element and it is located on the downstream air flow side of the air filter.

The air freshener filter insert strips are easy to install using conventional filter installation techniques.

For instance the cowl type of filter shown in FIG. 13 can be replaced by opening the vehicle hood, removing the screws holding the cowl cover, removing the weather stripping, removing the cowl cover, unsnapping the tabs holding the cover onto the housing, removing the filter frame, replacing the filter with a new air filter and inserting an air freshener filter insert strip with the projections containing the air freshening substance being disposed between the pleats of the air filter. Replacing the frame with the air filter and air freshener filter insert strip therein into the housing and attaching the cover with screws or snaps, snapping the cover onto the housing, replacing the weather stripping, and replacing the cowl cove with the screws before closing the hood.

As shown in FIG. 15, the air filter can be replaced and the filter freshener strip installed by simply opening the glove compartment, lifting up a cover panel at the rear of the compartment, opening the access door to the filter, removing the filter, replacing the filter with a new conventional pleated filter together with a freshener strip of the present invention nested or mounted by suitable means thereon, closing the access door, reattaching the cover panel and closing the glove compartment.

The aforementioned filter installation procedures can be followed to change most cabin air filters. Of course, depending upon the brand and/or type of vehicle additional steps may be required to replace the filter.

It is also contemplated that a fragrant strip can be bonded, adhesively attached, surrounded by a gasket and formed integrally with a conventional foam and/or fiber air particulate filter on the downstream side, sized and shaped to fit in the existing filter housing and disposed in the existing bracket or frame. For instance FIG. 17 shows the embodiment of FIG. 1, wherein solid air freshener filter insert particles 29 such as beads, flakes, bars, or grains are embedded in the porous foam material attached to the fibrous filter. The solid particles may be of any form or shape or be formed of small granular particles of a fragrant absorbing material such as an acetate polymer or one of the aforementioned derivatives thereof wherein the solid particulate material is impregnated with a time release substance of selected fragrance. The entire filter and air freshener filter insert can be packaged in an air-tight sealed container or plastic bag ready for removal and insertion into a conventional filter housing of a air ventilation system without requiring modification of the filter housing.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

We claim:

1. In a motor vehicle cabin air circulating system that has an airflow passage the improvement comprising providing an air flow through substrate having an air freshening substance releasably retained therein and locating the same across said airflow passage at a location between an inlet to said airflow passage and an outlet therefrom and wherein said outlet is located in the cabin of said vehicle.

2. An air flow through air freshening member for a vehicle cabin air circulating system comprising a base strip of porous material, a selected quantity of particulate material having an air freshening substance releasably retained therein, and means retaining said particulate material on said base strip.

3. The air flow through air freshening member for a vehicle cabin air circulating system defined in claim 2 wherein said particulate material is located in selected areas spaced apart from one another on said base strip.

4. The air flow through air freshening member for a vehicle cabin air circulating system as defined in claim 3 wherein said selected spaced apart areas are enlargements in said base strip.

5. The air flow through air freshening member for a vehicle cabin air circulating system as defined in claim 4 wherein said enlargements are pockets.

6. The air flow through air freshening member for a vehicle cabin air circulating system as defined in claim 5 wherein said pockets are integrally formed with said base strip.

7. The air flow through air freshening member for a vehicle cabin air circulating system as defined in claim 5 wherein said pockets are separate members and means attaching said pocket members to said base strip at positions spaced apart from one another longitudinally there along.

8. The air flow through air freshening member for a vehicle cabin air circulating system as defined in claim 5 wherein each said pocket is elongate and extending in a direction cross-wise of said base strip.

9. The air flow through air freshening member for a vehicle cabin air circulating system as defined in claim 8 wherein said elongate pockets extend in a direction transverse relative to the length of said base strip.

10. The air flow through air freshening member for a vehicle cabin air circulating system as defined in claim 8 wherein said pockets are spaced apart a selected distance from one another in a direction lengthwise of said base strip.

11. The air flow through air freshening member for a vehicle cabin air circulating system as defined in claim 2 wherein said particulate material is sufficiently coarse as to allow air of the air circulating system to readily flow there through.

12. The air flow through air freshening member for a vehicle cabin air circulating system as defined in claim 11 wherein said particulate material comprises beads of an acetate material.

13. The air flow through air freshening member for a vehicle cabin air circulating system as defined in claim 12 wherein said beads are impregnated with a slow time release substance having a preselected fragrance.

14. The air flow through air freshening member for a vehicle cabin air circulating system as defined in claim 2 wherein said base strip is an open mesh.

15. The air flow through air freshening member for a vehicle cabin air circulating system as defined in claim 14 wherein said base strip is made of a synthetic material.

16. The air flow through air freshening member for a vehicle cabin air circulating system as defined in claim 15 wherein said synthetic material is a polymer selected from the group comprising polyesters, fiberglass, polyethylene, nylon and combinations thereof.

17. An air flow through air freshening member for a motor vehicle cabin air circulating system comprising a longitudinal strip of porous material having sufficient porosity as to allow air of said air circulating system to pass there through, a selected quantity of particulate material having an air freshening substance releasably. retained therein, and means retaining said particulate material on said base strip of material.

18. The air flow through air freshening member for a vehicle cabin air circulating system as defined in claim 17 wherein said particulate retaining means comprises pockets captively retaining said particulate material on said base strip of material.

19. The air flow through air freshening member for a vehicle cabin air circulating system as defined in claim 18 wherein said pockets are spaced apart from one another longitudinally along said strip.

20. A method of servicing a vehicle forced air ventilating system having an airflow passage with an air filter therein comprising removing said filter, providing a replacement filter, positioning a selected length of an air filter freshener member defined in claims 17 or 18 on a face of said replacement filter and replacing the removed air filter with said replacement filter having the air freshener member mounted thereon.

21. In an air circulating system that has an airflow passage and conventional particulate filter disposed in a filter housing, an air freshener filter insert comprising:
an air flow through substrate for holding a solid material impregnated with a time release substance of selected fragrance for controlled release over a selected time period, said insert being positioned across said airflow passage at a location between an inlet to said airflow passage downstream of said particulate filter and an outlet therefrom, and wherein said outlet is located in the air flow ventilation system.
